(19) United States
(12) Patent Application Publication
(10) Pub. No.: US 2007/0219250 A1
(43) Pub. Date: Sep. 20, 2007

(54) Pharmaceutical Compositions of Nateglinide

(76) Inventors: Romi Singh, Uttar Pradesh (IN); Anu Shilpa, Delhi (IN); Naga Vishnubhotla Prasad, Andhra Pradesh (IN); Sanjeev Kumar Sethi, Haryana (IN)

Correspondence Address: RANBAXY INC., 600 COLLEGE ROAD EAST, SUITE 2100, PRINCETON, NJ, 08540, US

(21) Appl. No.: 10/596,013

(22) PCT Filed: Nov. 24, 2004
(86) PCT No.: PCT/IB04/03863

§ 371 Date: Jan. 22, 2007

(30) Foreign Application Priority Data
Nov. 28, 2003 (IN) 1497/DEL/2003

(51) Int. Cl. A61K 31/195; A61K 31/155; A61K 31/4439

(52) U.S. Cl. 514/342; 514/563; 514/636

(57) ABSTRACT

The present invention relates to pharmaceutical compositions comprising nateglinide in combination with a surfactant, and processes for their preparation.

### FIELD OF THE INVENTION

The present invention relates to pharmaceutical compositions comprising nateglinide in combination with a surfactant, and processes for their preparation.

### BACKGROUND OF THE INVENTION

Nateglinide is an amino acid derivative that lowers blood glucose levels by stimulating insulin secretion from the pancreas. It is widely indicated as monotherapy to lower blood glucose in patients with Type 2 diabetes and is described, for example, in EP 196,222 and EP 526,171. It is also indicated for use in combination with other anti-diabetic compounds, such as metformin. Nateglinide is available as oral tablets in 60 mg and 120 mg strengths, marketed in the US by Novartis under the trade name Starlix®.

Nateglinide is insoluble in water. This physical property creates unpredictable dissolution rates and leads to absorption problems.

U.S. Pat. No. 6,559,188 describes compositions of nateglinide or a pharmaceutically acceptable salt thereof wherein lactose and microcrystalline cellulose are used as fillers alone or in combination.

US 2003/0021843 discloses an antidiabetic preparation for oral administration containing nateglinide and at least one material selected from the group consisting of polysaccharides, polyacrylic acids, polylactic acids, polyoxyethylene, polyvinyl pyrrolidone, polyvinyl alcohol, oils and surfactants, nateglinide being dispersed in the material or being emulsified or microencapsulated with the material. In this application, the combination of oil and surfactant is used for the preparation of emulsions.

The use of surfactants in pharmaceutical formulations to assist in disintegration and dissolution of drug material is well known. Lachman et al. in Theory and Practice of Industrial Pharmacy, second edition, page 108-9, discloses the use of surface active agents or surfactants in almost every dosage form including liquids, semi-solids and solids. The surfactants play an important role in the absorption and efficacy of certain drugs.

In the present invention we have found that when nateglinide is combined with a surfactant, it leads to a surprising and unexpected discovery of use of surfactants to enhance the solubility and dissolution of solid dose oral formulations of poorly soluble drugs like nateglinide.

The present invention provides more flexibility and choice of pharmaceutical excipients like binders and fillers.

### SUMMARY OF THE INVENTION

In one general aspect there is provided an oral solid composition that includes nateglinide or pharmaceutically acceptable salts thereof and at least one pharmaceutically acceptable surfactant.

Embodiments of the solid composition may include one or more of the following features. For example, the surfactant may be one or more of anionic, nonionic, cationic, and mixtures thereof.

The anionic surfactant may be one or more of sodium lauryl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, and the like.

The nonionic surfactant may be one or more of polysorbate 80, nonyl phenol polyoxyethylene ether, tridecyl alcohol polyoxyethylene ether, dodecyl mercaptan polyoxyethylene thioether, the lauric ester of polyethylene glycol, the lauric ester of sorbitan polyoxyethylene ether or tertiary alkyl amine oxide, and the like.

The cationic surfactant may be one or more of distearyl dimethyl ammonium chloride, stearyl dimethyl benzyl ammonium chloride, stearyl trimethyl ammonium chloride, coco dimethyl benzyl ammonium chloride, dicoco dimethyl ammonium chloride, cetyl pyridinium chloride, cetyl trimethyl ammonium bromide, stearyl amine salts that are soluble in water such as stearyl amine acetate and stearyl amine hydrochloride, stearyl dimethyl amine hydrochloride, distearyl amine hydrochloride, alklyl phenoxyethoxyethyl dimethyl ammonium chloride, decyl pyridinium bromide, pyridinium chloride derivative of the acetyl amino ethyl esters of lauric acid, lauryl trimethyl ammonium chloride, decyl amine acetate, lauryl dimethyl ethyl ammonium chloride, the lactic acid and citric acid and other acid salts of stearyl-1-amidoimidazoline with methyl chloride, benzyl chloride, chloroacetic acid, and mixtures thereof.

Embodiments of the solid composition may also include one or more pharmaceutically acceptable excipients. The one or more pharmaceutically acceptable excipients may include fillers, binders, disintegrants, lubricants, glidants, coloring agents, flavoring agents and coatings.

The solid composition may also include at least one other anti-diabetic compound. The antidiabetic compound may be glitazones, sulfonyl urea derivatives and metformin, either in free form or in form of a pharmaceutically acceptable salt.

The solid composition may be in the form of one or more of a powder, tablet, granule, pellet, spheroid, caplet or capsule. The composition may be coated with a functional and/or non-functional film forming polymer.

In another general aspect there is provided a process for the preparation of an oral solid composition of nateglinide. The process includes the steps of: (a) blending nateglinide or pharmaceutically acceptable salts thereof, a surfactant and one or more pharmaceutically acceptable excipients; and (b) processing into a suitable solid dosage form.

Embodiments of the process may include one or more of the following features or those described above. For example, the blend of step (a) may be granulated. The granulation may be carried out by a wet granulation or a dry granulation technique. The blend may also be directly compressed. The wet granulation may be carried out using a granulating fluid. The granulating fluid may include one or more of methylene chloride, isopropyl alcohol, acetone, methanol, ethanol, water, and mixtures thereof. The dry granulation may be carried out by slugging or roller compaction.

The one or more pharmaceutically acceptable excipients may include fillers, binders, disintegrants, lubricants, glidants, coloring agents, flavoring agents and coatings.

The process may also include at least one other anti-diabetic compound. The antidiabetic compound may be glitazones, sulfonyl urea derivatives and metformin, either in free form or in form of a pharmaceutically acceptable salt.

Tablets produced by the process may be coated with one or more functional and/or non-functional layers.

In yet another general aspect there is provided a method for the treatment of metabolic disorders, type 2 diabetes mellitus, or a disease or condition associated with diabetes mellitus. The method includes administering to a patient in need thereof a pharmaceutical composition that includes nateglinide or pharmaceutically acceptable salts thereof; and at least one pharmaceutically acceptable surfactant.

The details of one or more embodiments of the inventions are set forth in the description below. Other features, objects and advantages of the inventions will be apparent from the description and claims.

### DETAILED DESCRIPTION OF THE INVENTION

The term ‘nateglinide’ as used herein includes nateglinide in a free or pharmaceutically acceptable salt form, in crystalline or amorphous form. For example, the nateglinide may be the B- or H-type crystal modification. The active ingredient or a pharmaceutically acceptable salt thereof may also be used in form of a hydrate or solvates thereof.

The amount of nateglinide to be used may vary from about 5% to about 70% (w/w), and in particular, from about 15% to about 40% (w/w), of the total pharmaceutical composition.

The term ‘surfactants’ as used herein includes a substance that lowers the surface tension of the medium in which it is dissolved, and/or the interfacial tension with other phases, and, accordingly, is positively adsorbed at the liquid/vapor and/or at other interfaces. Suitable surfactants include one or more of anionic, nonionic, cationic, and mixtures thereof.

The anionic surfactant is the reaction product of an organic compound, such as a high molecular weight acid or alcohol with an inorganic compound, such as sodium hydroxide or sulfuric acid, yielding a product wherein the organic part of the molecule, or the water-insoluble part of the molecule, has a negative charge and the water-soluble part of the molecule wherein the sodium ion has a positive charge.

The nonionic surfactants have a hydrophobic/hydrophilic balance wherein there is neither a negative nor a positive charge in either part of the molecule, thus giving it the nonionic terminology.

The cationic surfactants are formed in reactions where alkyl halides react with primary, secondary, or tertiary fatty amines. Here, the water-insoluble part of the molecule has a positive charge and the water-soluble part of the molecule is negatively charged, thus giving it the name of a cationic surface-active agent. Cationic surface-active agents reduce surface tension and are used as wetting agents in acid media.

The amount of surfactant to be used may vary from about 0.5% to about 10% (w/w), and in particular, from about 1% to about 5% (w/w), of the total pharmaceutical composition.

The term “solid composition” as used herein includes solid dosage forms, for example powder, tablet, granule, pellet, spheroid, caplet or capsule, and the like.

The term ‘composition’ as used herein may include other pharmaceutically acceptable excipients routinely used in the art of manufacturing pharmaceutical dosage forms. For example, the pharmaceutically acceptable excipients include one or more of fillers, binders, disintegrants, lubricants, glidants, coloring agents, flavoring agents and coatings.

Suitable fillers include one or more of corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powdered, dextrates, dextrins, dextrose, fructose, kaolin, lactitol, mannitol, starch, and starch pregelatinized.

Suitable binders include one or more of methyl cellulose, hydroxypropyl cellulose, polyvinylpyrrolidone, gelatin, gum Arabic, ethyl cellulose, polyvinyl alcohol, pullulan, pregelatinized starch, agar, tragacanth, sodium alginate, and propylene glycol.

Suitable disintegrants include one or more of starch, croscarmellose sodium, crospovidone, and sodium starch glycolate.

Suitable lubricants and glidants include one or more of colloidal anhydrous silica, stearic acid, magnesium stearate, calcium stearate, talc, hydrogenated castor oil, sucrose esters of fatty acids, microcrystalline wax, yellow beeswax, and white beeswax.

Suitable coloring agents include one or more FDA approved colors for oral use.

The compositions of nateglinide may be prepared by processes known in the prior art including comminuting, mixing, granulation, melting, sizing, filling, drying, molding, immersing, coating, compressing, extrusion-spheronization, etc.

The oral solid composition of nateglinide may be prepared by processes, for example, wet granulation, dry granulation or direct compression and may be in the form of tablets or capsules.

The process of direct compression may include preparing a blend of nateglinide, surfactant, filler, disintegrant, binder, lubricant and glidant; and compressing the blend into a tablet.

The process of dry granulation may be carried out by slugging or roller compaction. The composition of nateglinide may be prepared by the process of blending nateglinide, surfactant, filler, disintegrant and binder; compacting or slugging the blend; breaking the slugs to make granules; lubricating and compressing the lubricated granules.

The process of wet granulation may be carried out by blending nateglinide, surfactant, filler, and disintegrant; and granulating the blend with a solution/dispersion of the binder. Alternatively, the binder is added to the above blend and the resulting blend is granulated with a suitable solvent. The granules are dried and may be mixed with other excipients like disintegrant, lubricant, glidant and colors and compressed into tablets. The granulation may also be carried out in a fluidized bed dryer and sizing may be done by milling or pulverizing.

In one embodiment, the composition of nateglinide may be prepared by blending nateglinide, surfactant, filler, disintegrant and glidant; granulating the blend with a binder solution; drying and sizing the granules; mixing with a disintegrant; lubricating and compressing the lubricated granules.

In another embodiment, the composition of nateglinide may be prepared by blending nateglinide, surfactant, filler, disintegrant, binder and glidant; granulating the blend with a solvent; drying and sizing the granules; mixing with a disintegrant; lubricating and compressing the lubricated granules.

The blend of nateglinide and surfactant may be further mixed with one or more anti-diabetic compound prior to granulation. Suitable compounds include one or more of glitazones, sulfonyl urea derivatives and metformin. These compounds may be in free form or in the form of a pharmaceutically acceptable salt.

The tablets prepared by the present invention may be coated with one or more additional layers of film forming agents and/or pharmaceutically acceptable excipients.

The coating layers over the tablet may be applied as solution/dispersion of coating ingredients using any conventional technique known in the prior art such as spray coating in a conventional coating pan or fluidized bed processor; and dip coating.

Suitable solvents used for preparing a solution/dispersion of the coating ingredients include methylene chloride, isopropyl alcohol, acetone, methanol, ethanol, water and mixtures thereof.

Suitable film forming agents include one or more of ethyl cellulose, hydroxypropyl methylcellulose, hydroxypropyl cellulose, methyl cellulose, carboxymethylcellulose, hydroxymethylcellulose, hydroxyethylcellulose, hydroxypropyl methyl phthalate, cellulose acetate, cellulose acetate trimelliatate, cellulose acetate phthalate; Waxes such as polyethylene glycol; methacrylic acid polymers such as Eudragit® RL and RS; and the like and mixture thereof. Alternatively, commercially available coating compositions comprising film-forming polymers marketed under various trade names, such as Opadry® may also be used for coating.

The following examples are illustrative of the invention, and are not to be construed as limiting the invention.

### EXAMPLE 1

| | Quantity |
|---|---|
| Ingredient | (wt/tablet) mg |
| Nateglinide | 121.21* |
| Lactose | 424.16 |
| Povidone | 12 |
| Croscarmellose sodium | 20 |
| Colloidal silicon dioxide | 16 |
| Purified water | q.s |
| Croscarmellose Sodium | 12.8 |
| Colloidal silicon dioxide | 12.8 |
| Magnesium stearate | 11.4 |

*Equivalent to Nateglinide 120 mg after potency and moisture adjustment

Process:
1. Nateglinide, lactose, povidone, colloidal silicon dioxide and a part of croscarmellose sodium are mixed in a high shear mixer and granulated using purified water.
2. The wet granules are dried in a fluid bed drier, passed through a screen and then sized.
3. The colloidal silicon dioxide and the rest of the croscarmellose sodium are mixed, passed through a screen and blended with the granules of step 2.
4. The magnesium stearate is passed through a screen, blended with the blend of step 3 and the resulting mixture is compressed to tablets.

### EXAMPLE 2

| | Quantity |
|---|---|
| Ingredient | (wt/tablet) mg |
| Nateglinide | 121.21* |
| Lactose | 343.79 |
| Sodium lauryl sulphate | 12.5 |
| Povidone | 12 |
| Croscarmellose sodium | 20 |
| Colloidal silicon dioxide | 16 |
| Purified water | q.s |
| Croscarmellose Sodium | 12.8 |
| Colloidal silicon dioxide | 12.8 |
| Magnesium stearate | 11.4 |

*Equivalent to Nateglinide 120 mg after potency and moisture adjustment

Process:
1. Nateglinide, lactose, sodium lauryl sulphate, povidone, colloidal silicon dioxide and a part of croscarmellose sodium are mixed in a high shear mixer and granulated using purified water.
2. The wet granules are dried in a fluid bed drier, passed through a screen and then sized.
3. The colloidal silicon dioxide and the rest of the croscarmellose sodium are mixed, passed through a screen and blended with the granules of step 2.
4. The magnesium stearate is passed through a screen, blended with the blend of step 3 and the resulting mixture is compressed to tablets.

### EXAMPLE 3

| | Quantity |
|---|---|
| Ingredient | (wt/tablet) mg |
| Nateglinide | 121.21* |
| Lactose | 343.79 |
| Polysorbate 80 | 12.5 |
| Povidone | 12 |
| Croscarmellose sodium | 20 |
| Colloidal silicon dioxide | 16 |
| Purified water | q.s |
| Croscarmellose Sodium | 12.8 |
| Colloidal silicon dioxide | 12.8 |
| Magnesium stearate | 11.4 |

*Equivalent to Nateglinide 120 mg after potency and moisture adjustment

Process:
1. Nateglinide, lactose, polysorbate 80, povidone, colloidal silicon dioxide and a part of croscarmellose sodium are mixed in a high shear mixer and granulated using purified water.
2. The wet granules are dried in a fluid bed drier, passed through a screen and then sized.
3. The colloidal silicon dioxide and the rest of the croscarmellose sodium are mixed, passed through a screen and blended with the granules of step 2.
4. The magnesium stearate is passed through a screen, blended with the blend of step 3 and the resulting mixture is compressed to tablets.

### EXAMPLE 4

| | Quantity |
|---|---|
| Ingredient | (wt/tablet) mg |
| Nateglinide | 120 |
| Microcrystalline cellulose | 425 |
| Povidone | 12 |
| Croscarmellose sodium | 20 |
| Colloidal silicon dioxide | 16 |
| Purified water | q.s |
| Croscarmellose Sodium | 12.8 |
| Colloidal silicon dioxide | 12.8 |
| Magnesium stearate | 11.4 |

Process:
1. Nateglinide, microcrystalline cellulose, povidone, colloidal silicon dioxide and a part of croscarmellose sodium are mixed in a high shear mixer and granulated using purified water.
   2. The wet granules are dried in a fluid bed drier, passed through a screen and then sized.
   3. The colloidal silicon dioxide and the rest of the croscarmellose sodium are mixed, passed through a screen and blended with the granules of step 2.
   4. The magnesium stearate is passed through a screen, blended with the blend of step 3 and the resulting mixture is compressed to tablets.

### EXAMPLE 5

| | Quantity |
|---|---|
| Ingredient | (wt/tablet) mg |
| Nateglinide | 120 |
| Microcrystalline cellulose | 412.5 |
| Sodium lauryl sulphate | 12.5 |
| Povidone | 12 |
| Croscarmellose sodium | 10 |
| Colloidal silicon dioxide | 16 |
| Purified water | q.s |
| Croscarmellose Sodium | 22.8 |
| Colloidal silicon dioxide | 12.8 |
| Magnesium stearate | 11.4 |

Process:
1. Nateglinide, microcrystalline cellulose, sodium lauryl sulphate, povidone, colloidal silicon dioxide and a part of croscarmellose sodium are mixed in a high shear mixer and granulated using purified water.
2. The wet granules are dried in a fluid bed drier, passed through a screen and then sized.
3. The colloidal silicon dioxide and the rest of the croscarmellose sodium are mixed, passed through a screen and blended with the granules of step 2.
4. The magnesium stearate is passed through a screen, blended with the blend of step 3 and the resulting mixture is compressed to tablets.

### EXAMPLE 6

| | Quantity |
|---|---|
| Ingredient | (wt/tablet) mg |
| Nateglinide | 120 |
| Microcrystalline cellulose | 412.5 |
| Polysorbate 80 | 12.5 |
| Povidone | 12 |
| Croscarmellose sodium | 10 |
| Colloidal silicon dioxide | 16 |
| Purified water | q.s |
| Croscarmellose Sodium | 22.8 |
| Colloidal silicon dioxide | 12.8 |
| Magnesium stearate | 11.4 |

Process:
1. Nateglinide, microcrystalline cellulose, polysorbate 80, povidone, colloidal silicon dioxide and a part of croscarmellose sodium are mixed in a high shear mixer and granulated using purified water.
2. The wet granules are dried in a fluid bed drier, passed through a screen and then sized.
3. The colloidal silicon dioxide and the rest of the croscarmellose sodium are mixed, passed through a screen and blended with the granules of step 2.
4. The magnesium stearate is passed through a screen, blended with the blend of step 3 and the resulting mixture is compressed to tablets.
Comparative In Vitro Dissolution Study
In vitro release profile of nateglinide prepared according to Examples 1-6 was studied in 1000 ml, 0.01 N HCl, with 0.5% SLS (pH-1.2), using USP apparatus—II, at 50 rpm. The results are provided in Table 1.

Table 1: In vitro release profile of nateglinide prepared according to Examples 1-6.

| | Cumulative percentage (%) release of nateglinidie from | | | | | | |
|---|---|---|---|---|---|---|---|
| Time | Tablets (w/w) | | | | | | |
| (min.) | Starlix ® | Example 1 | Example 2 | Example 3 | Example 4 | Example 5 | Example 6 |
| 10 | 62 | 40 | 50 | 54 | — | — | — |
| 15 | — | — | — | — | 38 | 51 | 66 |
| 20 | — | 43 | — | 71 | — | — | — |
| 30 | 65 | 72 | 71 | 80 | 46 | 67 | 80 |
| 45 | 67 | 77 | 81 | 87 | 54 | 75 | 87 |
| 60 | 72 | — | — | — | 69 | — | — |
| Infinity | 93 | 96 | 98 | 96 | — | 96 | 96 |

As can be seen from the data above, a formulation that includes a surfactant (Example 2, 3, 5 and 6) shows a better dissolution profile as compared to formulations without a surfactant (Example 1 and 4).

### EXAMPLE 7

| | |
|---|---|
| | Quantity |
| Ingredient | (wt/tablet) mg |

Intragranular

| | |
|---|---|
| Nateglinide | 120.84 |
| Lactose Monohydrate | 325 |
| Microcrystalline cellulose | 87.16 |
| Povidone | 12 |
| Croscarmellose sodium | 20 |
| Colloidal silicon dioxide | 28 |
| Purified water | q.s |

Extragranular

| | |
|---|---|
| Croscarmellose Sodium | 12.8 |
| Colloidal silicon dioxide | 12.8 |
| Magnesium stearate | 11.4 |
| Total weight | 630.0 |

Process:
1. Nateglinide is blended with lactose monohydrate, microcrystalline cellulose, colloidal silicon dioxide and croscarmellose sodium.
2. Povidone is dissolved in purified water and the solution is used to granulate the blend obtained in step 1.
3. The wet granules are dried, passed through a screen and then sized.
4. The colloidal silicon dioxide and croscarmellose sodium are mixed, passed through a screen and blended with the granules of step 3.
5. The magnesium stearate is passed through a screen, blended with the blend of step 4 and the resulting mixture is compressed to tablets.

### EXAMPLE 8

| | |
|---|---|
| | Quantity |
| Ingredient | (wt/tablet) mg |

Intragranular

| | |
|---|---|
| Nateglinide | 121.21* |
| Lactose Monohydrate | 325 |
| Microcrystalline cellulose | 84.79 |
| Sodium lauryl sulphate | 12 |
| Povidone | 12 |
| Croscarmellose sodium | 20 |
| Colloidal silicon dioxide | 28 |
| Purified water | q.s |

Extragranular

| | |
|---|---|
| Croscarmellose Sodium | 12.8 |
| Colloidal silicon dioxide | 12.8 |
| Magnesium stearate | 11.4 |
| Total weight | 640.0 |

*d₉₀ = 54 μm; d₅₀ = 15 μm

Process:
1. Nateglinide is blended with lactose monohydrate, microcrystalline cellulose, colloidal silicon dioxide and croscarmellose sodium.
2. Povidone and sodium lauryl sulphate are dissolved in purified water and the solution is used to granulate the blend obtained in step 1.
3. The wet granules are dried, passed through a screen and then sized.
4. The colloidal silicon dioxide and croscarmellose sodium are mixed, passed through a screen and blended with the granules of step 3.
5. The magnesium stearate is passed through a screen, blended with the blend of step 4 and the resulting mixture is compressed to tablets.

### EXAMPLE 9

| | |
|---|---|
| | Quantity |
| Ingredient | (wt/tablet) mg |

Intragranular

| | |
|---|---|
| Nateglinide | 121.21* |
| Lactose Monohydrate | 325 |
| Microcrystalline cellulose | 84.79 |
| Sodium lauryl sulphate | 12 |
| Povidone | 12 |
| Croscarmellose sodium | 20 |
| Colloidal silicon dioxide | 28 |
| Purified water | q.s |

Extragranular

| | |
|---|---|
| Croscarmellose Sodium | 12.8 |
| Colloidal silicon dioxide | 12.8 |
| Magnesium stearate | 11.4 |
| Total weight | 640.0 |

*d₉₀ = 6 μm; d₅₀ = 2 μm

Process: Same as in Example 8.

### EXAMPLE 10

| | |
|---|---|
| | Quantity |
| Ingredient | (wt/tablet) mg |

Intragranular

| | |
|---|---|
| Nateglinide | 120.24 |
| Lactose Monohydrate | 324.76 |
| Microcrystalline cellulose | 86 |
| Sodium lauryl sulphate | 12 |
| Povidone | 12 |
| Croscarmellose sodium | 20 |
| Colloidal silicon dioxide | 28 |
| Purified water | q.s |

Extragranular

| | |
|---|---|
| Croscarmellose Sodium | 12.8 |
| Colloidal silicon dioxide | 12.8 |
| Magnesium stearate | 11.4 |
| Total weight | 640.0 |

Process:
1. Nateglinide is blended with lactose monohydrate, microcrystalline cellulose, povidone, colloidal silicon dioxide and croscarmellose sodium.
2. Sodium lauryl sulphate is dispersed in purified water and the dispersion is used to granulate the blend obtained in step 1.
3. The wet granules are dried, passed through a screen and then sized.
4. The colloidal silicon dioxide and croscarmellose sodium are mixed, passed through a screen and blended with the granules of step 3.
5. The magnesium stearate is passed through a screen, blended with the blend of step 4 and the resulting mixture is compressed to tablets.
Comparative In Vitro Dissolution Study

In vitro release profile of nateglinide prepared according to Examples 7-10 was studied in 1000 ml, 0.01 N HCl, with 0.5% SLS (pH-1.2), using USP apparatus—II, at 50 rpm. The results are provided in Table 2.

TABLE 2
In vitro release profile of nateglinide prepared
according to Examples 7-10.

| | Cumulative percentage (%) release of | | | |
|---|---|---|---|---|
| Time | nateglinide from Tablets (w/w) | | | |
| (min.) | Example 7 | Example 8 | Example 9 | Example 10 |
| 10 | 47 | 54 | 47 | 87 |
| 20 | 59 | 69 | 77 | 93 |
| 30 | 68 | 78 | 85 | 97 |
| 45 | 76 | 87 | 95 | 97 |
| (Infinity) | | | | |

As can from the data above, a formulation that includes a surfactant (Example 8, 9 and 10) shows a better dissolution profile as compared to a formulation without a surfactant (Example 7).

While the present invention has been described in terms of its specific embodiments, certain modifications and equivalents will be apparent to those skilled in the art and are included within the scope of the present invention.

1. An oral solid composition of nateglinide comprising:
a) nateglinide or pharmaceutically acceptable salts thereof; and
b) at least one pharmaceutically acceptable surfactant,

2. The oral solid composition of claim 1, wherein the nateglinide comprises an amount of from about 5% w/w to about 70% w/w of the composition.

3. The oral solid composition of claim 1, wherein the surfactant comprises one or more of anionic, nonionic, cationic, and mixtures thereof.

4. The oral solid composition of claim 3, wherein the anionic surfactants comprises one or more of sodium lauryl sulphate, potassium dodecyl sulphonate, sodium dodecyl benzene sulphonate, sodium salt of lauryl polyoxyethylene sulphate, lauryl polyethylene oxide sulfonate, dioctyl ester of sodium sulphosuccinic acid or sodium lauryl sulphonate, and mixtures thereof.

5. The oral solid composition of claim 4, wherein the surfactant is sodium lauryl sulphate.

6. The oral solid composition of claim 3, wherein the nonionic surfactants comprises one or more of polysorbate 80, nonyl phenol polyoxyethylene ether, tridecyl alcohol polyoxyethylene ether, dodecyl mercaptan polyoxyethylene thioether, the lauric ester of polyethylene glycol, the lauric ester of sorbitan polyoxyethylene ether or tertiary alkyl amine oxide, and mixtures thereof.

7. The oral solid composition of claim 6, wherein the surfactant is polysorbate 80.

8. The oral solid composition of claim 3, wherein the cationic surfactants comprises one or more of distearyl dimethyl ammonium chloride, stearyl dimethyl benzyl ammonium chloride, stearyl trimethyl ammonium chloride, coco dimethylbenzyl ammonium chloride, dicoco dimethyl ammonium chloride, cetyl pyridinium chloride, cetyl trimethyl ammonium bromide, stearyl amine salts that are soluble in water such as stearyl amine acetate and stearyl amine hydrochloride, stearyl dimethyl amine hydrochloride, distearyl amine hydrochloride, alkyl phenoxyethoxyethyl dimethyl ammonium chloride, decyl pyridinium bromide, pyridinium chloride derivative of the acetyl amino ethyl esters of lauric acid, lauryl trimethyl ammonium chloride, decyl amine acetate, lauryl dimethyl ethyl ammonium chloride, the lactic acid and citric acid and other acid salts of stearyl-1-amidoimidazoline with methyl chloride, benzyl chloride, chloroacetic acid and similar compounds, and mixtures thereof.

9. The solid composition of claim 1, wherein the surfactant comprises an amount of from about 0.5% w/w to about 10% w/w of the composition.

10. The oral solid composition of claim 1, wherein the composition further comprises one or more pharmaceutically acceptable excipients comprising fillers, binders, disintegrants, lubricants, glidants, coloring agents, flavoring agents, and coatings.

11. The oral solid composition of claim 10, wherein the filler comprises one or more of corn starch, lactose, white sugar, sucrose, sugar compressible, sugar confectioners, glucose, sorbitol, calcium carbonate, calcium phosphate-dibasic, calcium phosphate-tribasic, calcium sulfate, microcrystalline cellulose, silicified microcrystalline cellulose, cellulose powered, dextrates, dextrins, dextrose, fructose, kaolin, lactitol, mannitol, sorbitol, starch, starch pregelatinized, sucrose, and mixtures thereof.

12-13. (canceled)

14. The oral solid composition of claim 10, wherein the binder comprises one or more of methyl cellulose, hydroxypropyl, cellulose, polyvinylpyrrolidone, gelatin, gum arabic, ethyl cellulose, polyvinyl alcohol, pullulan, pregelatinized starch, agar, tragacanth, sodium alginate, propylene glycol, and mixtures thereof.

15. (canceled)

16. The oral solid composition of claim 10, wherein the disintegrant comprises one or more of starch, croscarmellose, sodium, crospovidone, sodium starch glycolate, and mixtures thereof.

17. (canceled)

18. The oral solid composition of claim 10, wherein the lubricant comprises one or more colloidal anhydrous silica, stearic acid, magnesium stearate, calcium stearate, talc, hydrogenated castor oil, sucrose esters of fatty acids, microcrystalline wax, yellow beeswax, white beeswax, and mixtures thereof.

19. (canceled)

20. The oral solid composition of claim 1, further comprising at least one other anti-diabetic compound.

21. The oral solid composition of claim 20, wherein the antidiabetic compound comprises glitazones, sulfonyl urea derivatives and metformin, either in free for or in form of a pharmaceutically acceptable salt thereof.

22. The oral solid composition of claim 1, wherein the composition comprises one or more of powder, tablets, granules, pellets, spheroids, caplets and capsules.

23-32. (canceled)

33. A process for the preparation of a pharmaceutical composition of nateglinide, the process comprising the steps of:
i. blending nateglinide or pharmaceutically acceptable salts thereof, surfactant and one or more pharmaceutically acceptable excipients; and;
ii. processing into a solid dosage form.

34. The process of claim 33, wherein the blend of step a) is granulated.

35. The process of claim 34, wherein the granulation is carried out by a wet granulation or a dry granulation technique.

36. (canceled)

37. The process of claim 35, wherein the wet granulation is carried out using a granulating fluid comprising one or more of methylene chloride, isopropyl alcohol, acetone, methanol, ethanol, water, and mixtures thereof.

38. (canceled)

39. The process of claim 35, wherein the dry granulation is carried out by slugging or roller compaction.

40. (canceled)

41. The process of claim 33, further comprising mixing at least one other antidiabetic compound.

42. The process of claim 41, wherein the antidiabetic compound comprises one or more of glitazones, sulfonyl urea derivatives and metformin, either in free form or in form of a pharmaceutically acceptable salt.

43. The process of claim 33, wherein the dosage form comprises one or more of powder, tablets, granules, pellets, spheroids, caplets and capsules.

45-46. (canceled)

47. A process for preparation of oral tablets of nateglinide, the process comprising blending nateglinide, surfactant, filler, disintegrant, binder and lubricant; and compressing.

48. A method for the prevention or treatment of metabolic disorders, type 2 diabetics mellitus, or disease or condition associated with diabetes mellitis, the method comprising administering to a patient in need thereof a pharmaceutical composition comprising nateglinide or pharmaceutically acceptable salts thereof; and at least one pharmaceutically acceptable surfactant.
